(19) United States
(12) Patent Application Publication
(10) Pub. No.: US 2004/0023893 A1
(43) Pub. Date: Feb. 5, 2004

(54) Novel coumarin derivatives and the salts thereof, a process for the preparation thereof and their use in the pharmaceutical field

(76) Inventors: Marco Prosdocimi, Padova (IT); Giampaolo Menon, Battaglia Terme (IT); Giovanni Monastra, Padova (IT); Enrico Galbiati, Abano Terme (IT); Mario Finesso, Montegrotto Terme (IT)

Correspondence Address: BIRCH STEWART KOLASCH & BIRCH, PO BOX 747, FALLS CHURCH, VA, 22040-0747, US

(21) Appl. No.: 10/343,241

(22) PCT Filed: Jul. 26, 2001
(86) PCT No.: PCT/EP01/08642

(30) Foreign Application Priority Data
Jul. 31, 2000 (IT) PD2000A000193

(51) Int. Cl. A61K 31/7048; A61K 31/452; A61K 31/4025

(52) U.S. Cl. 514/27; 514/456; 549/283; 514/217.03; 514/320; 514/422; 536/8; 540/598; 546/196; 548/525

(57) ABSTRACT

The present invention relates to novel coumarin derivatives of formula (I) wherein: X═O or S; n=zero, 1, 2, 3 or 4; R₅ and R₆, which can be the same or different, are optionally unsaturated C₁-C₄ alkyl groups, or together with the nitrogen atom they form a residue of cyclic amines optionally containing other heteroatoms; R₁═CH₃ or phenyl; R₂ and R [symbol] 4, which can be the same or different, are H, OH, allyl, halogen or methyl.

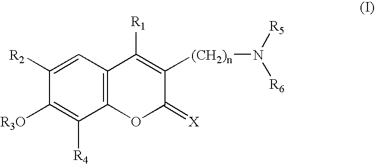

The present invention relates to novel coumarin derivatives of formula (I):

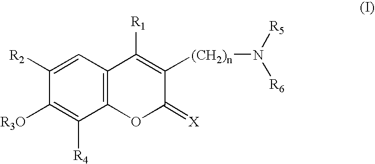

wherein:

X═O or S;

n=zero, 1, 2, 3 or4;

R₅ and R₆, which can be the same or different, are optionally unsaturated C₁-C₄ alkyl groups, or together with the nitrogen atom they form a residue of cyclic amines optionally containing other heteroatoms;

R₁═CH₃ or phenyl;

R₂ and R₄, which can be the same or different, are H, OH, allyl, halogen or methyl;

R₃═H, straight or branched, saturated or unsaturated C₁-C₁₀ alkyl, which can bear OH groups, amido groups, residues of simple or derivatized sugars, or residues of optionally derivatized amino acids, or can be an optionally branched alkylene chain, (“spacer”), which links together the two residues, which can be the same or different, of formula (II)

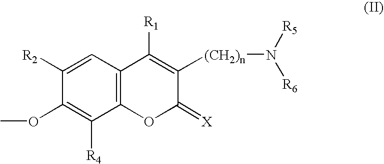

wherein X, n, R₁, R₂, R₄, R₅ and R₆ have the meanings defined above.

The invention also comprises the salts of said compounds, particularly those with pharmaceutically acceptable bases or acids, and the processes for the preparation thereof.

The novel coumarin derivatives and the salts according to the present invention have interesting pharmacological properties and may therefore be used to advantage to treat major pathologies such as peripheral ischaemia and organ ischaemia, electrical alterations of the myocardium and other organs resulting from the release of pro-inflammatory molecules (TNF, IL-1, NO, etc), peripheral and cerebral vasculopathies, angina-type disorders, hypercholesterolaemia, systemic infections such as sepsis, allergic pathologies such as asthma, rhinitis, eczema, dermatitis, such as antithrombotics and antihypertensives. The present invention also includes pharmaceutical preparations containing one or more of said derivatives or their salts in the form of capsules, tablets, injectable solutions, sprays and controlled release systems, creams, gels and transdermal systems.

### BACKGROUND OF THE INVENTION

Coumarins include a large class of phenol substances that are to be found in plants, and are constituted by a benzene ring and an α-pyrone ring fused together. To date, at least 1,300 coumarins have been identified, mainly as metabolites of green plants, in fungi and in bacteria.

Cloricromene (commercial name Proendotel) belongs to a family of coumarins and is prepared by the process described in U.S. Pat. Nos. 4,296,039 and 4,452,811.

Besides its known coronary vasodilatory and anti-arrhythmic properties (U.S. Pat. No. 4,349,566) and anti-platelet aggregation properties (U.S. Pat. No. 4,362,741), it has been seen that cloricromene is able to inhibit many of the cellular functions of polymorphonuclear leukocytes (Bertocchi et al.: Arch. Pharmacol. 1989, 339, 697-703; Gresele et al.: Biochem. Pharmacol. 1993, 45, 123-130) and may have positive effects on biochemical interactions between different cell species, in particular between platelets and polymorphonuclear leukocytes (Zatta et al.: Eur. J. Pharmacol. 1991, 198, 97-100), that are known to be relevant in thrombotic and ischaemic states.

Cloricromene also has a documented effect in models of ischaemia and reperfusion in various organs, in which inflammatory-type cytokines are also involved, such as TNF (Squadrito et al.: Life Sciences 1993, 53, 341-355), and its activity is also known in the treatment of pathologies linked with vasodilatory processes and tissue damage characterised by an increased production of nitrogen oxide (patent by the Applicant No. IT 1265665), such as, for example, pulmonary inflammation, oedema, erythema, dermatitis, psoriasis, skin ulcers, arthritis, rheumatoid arthritis and other autoimmune disorders, hypotensive shock, septic shock, hypovolemic shock, vasculitis such as inflammations consequent on thrombo-phlebitis, ulcerative colitis.

### DETAILED DESCRIPTION OF THE INVENTION

In view of these known properties of Cloricromene's and since cytokines are known to be strong pro-inflammatory agents, a series of compounds have been prepared and tested for their activity in vitro and in vivo on the synthesis of cytokines such as TNF, as well as for their action on other cellular phenomena, such as platelet aggregation and the production of free radicals, as well as their action in in vivo models of inflammation. Among the drugs currently used in inflammatory processes, the non-steroid compounds are known for their poor, or lack of, ability to inhibit the release of inflammatory cytokines, that are, on the contrary, inhibited by steroid-type compounds. Since these compounds have various toxic activities, the availability of non-steroid products specifically active on cytokine synthesis is particularly useful in developing innovative therapies.

The experimental results obtained have demonstrated that the compounds that are the subject of the present invention have activities that are better than and different from those of cloricromene. In particular, it was observed that some compounds have stronger actions than cloricromene on the synthesis of TNF, a known inflammatory cytokine, and they are not active in the process of platelet aggregation and the release of free radicals.

This increased selectivity of action makes the compounds of the present invention therapeutically advantageous.

Furthermore, in vivo characterisation has indicated that the compounds of the invention have lower acute toxicity than cloricromene, as demonstrated by the fact that toxic or lethal effects are observed only at higher dosages.

Therefore, due to the strict relationship between the free compounds and the salts of the present invention, wherever feasible, all that is indicated hereafter with regard to the free substances will be true also of their salts.

Preferred compounds of the present invention are the following:

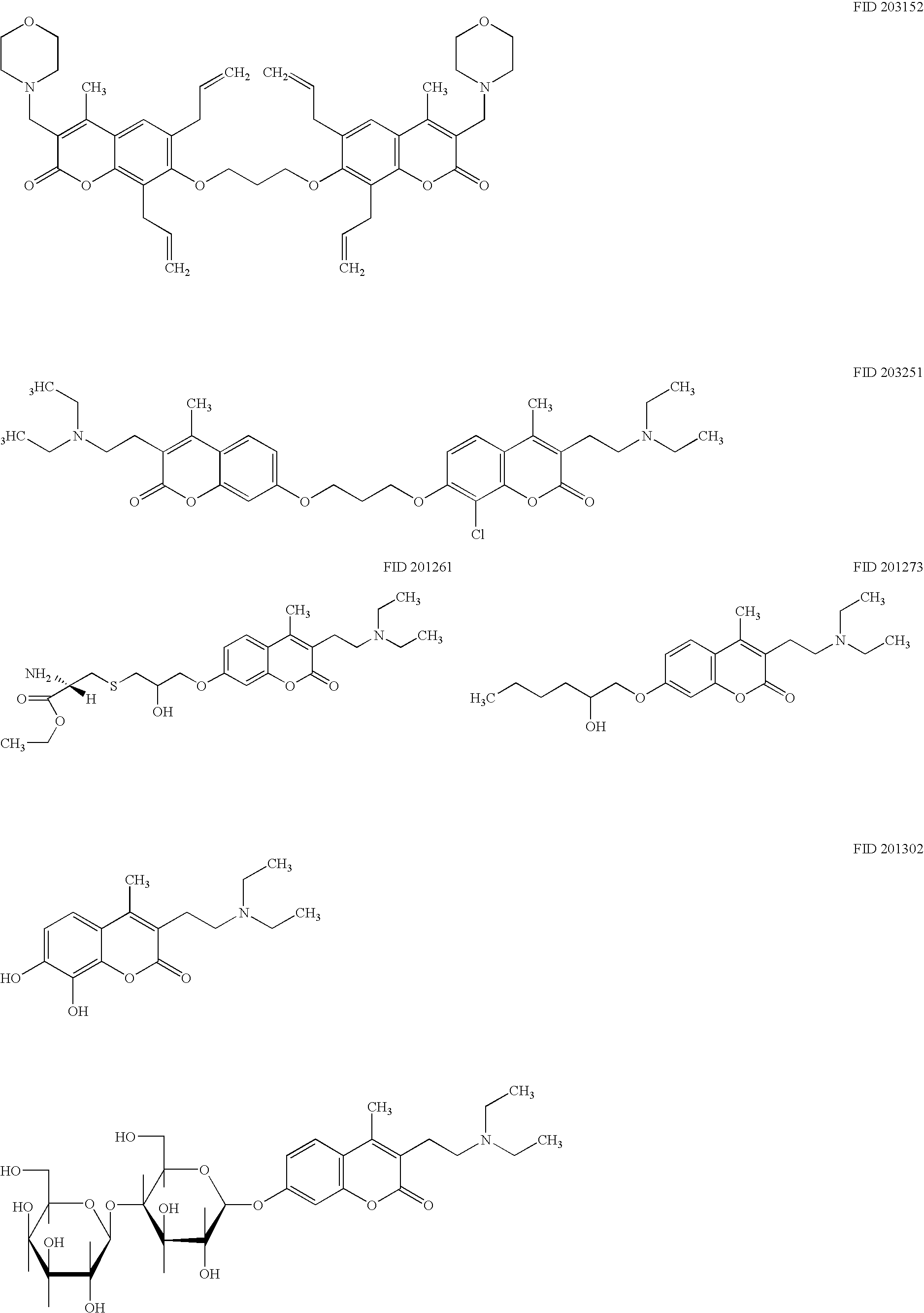

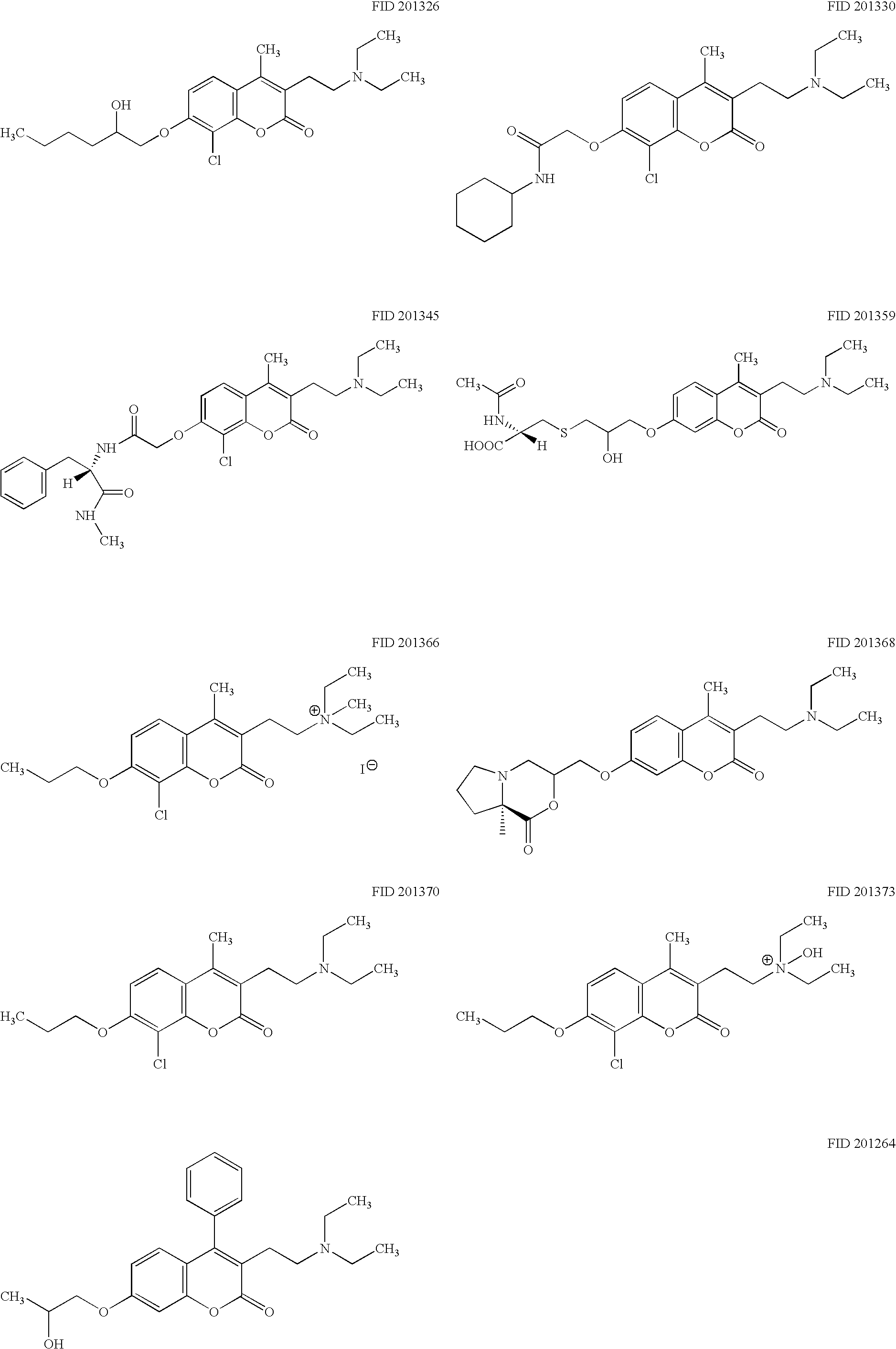

Some compounds of the present invention were tested in various in vitro and in vivo models:

inhibition of TNF release after stimulation with LPS in vitro

inhibition of TNF release after stimulation with LPS in vivo

reduced inflammation in carrageenin-induced oedema in rat paw

inhibition of nitrite-nitrate release in rat plasma

inhibition of superoxide anion formation induced by f-MLP in human whole blood

inhibition of platelet aggregation in human whole blood

acute toxicity after a single intravenous administration

mutagenesis (mini-Ames test)

Test 1—Inhibition of TNF Release After Stimulation with LPS in vitro

The test compound was added to the culture medium in a murine macrophage line (J774) or to whole blood anti-coagulated with heparin. The cells were then stimulated with bacterial lipopolysacccharide. After incubation at 37° C. for a suitable length of time, the supernatant was removed from the culture and incubated with a line of murine fibroblasts (L929), sensitive to TNF. The quantity of TNF released after stimulation with LPS is measured by comparing mortality of the L929 cells with that of the controls. Table 1 reports for the single compounds the concentration (±standard error) able to inhibit fibroblast mortality (L929) by 50%.

TABLE 1

| | J774 CI₅₀ | Human blood |
|---|---|---|
| Compound | (μM) | CI₅₀ (μM) |
| 201006 (Proendotel ®) | 57.9 ± 20 | 78.6 ± 10 |
| 203152 | 13.3 ± 9 | >80 |
| 201261 | 25.4 | 9 |
| 201273 | 27.5 ± 5.3 | 40.4 ± 13.5 |
| 201326 | 10.5 ± 3.0 | 23 ± 2.0 |
| 201330 | 12 ± 3.9 | 12.6 ± 3.5 |

Test 2—Inhibition of the Release of TNF and IL-β Following Stimulation with LPS in vivo

Conscious rats were injected with a dose of LPS, which is able to stimulate the release of TNF and IL-1β, having first received an intravenous administration (15 minutes beforehand) of the test compound at a dose of 0.5 mg/Kg or 2 mg/Kg. Blood levels of TNF and IL-1β were measured by the ELISA method on blood samples taken 75 minutes and 120 minutes after LPS respectively.

Table 2 below reports the percent of inhibition of each product compared to that of the controls treated with saline.

TABLE 2

| | Dose of 0.5 mg/Kg | | Dose of 2 mg/Kg | |
|---|---|---|---|---|
| | % of | % of | % of | % of |
| | inhibition of | inhibition of | inhibition of | inhibition of |
| Compound | TNF | IL-1β | TNF | IL-1β |
| 201006 | 9 | 45 | 30 | 28 |
| (Proendotel) | | | | |
| 203152 | 72 | 64 | 73 | 60 |
| 201273 | 72 | 67 | 84 | 39 |
| 201326 | 85 | 40 | 77 | 62 |
| 201330 | 75 | 33 | 62 | 57 |

Test 3—Reduction in Inflammation in Carrageenin-Induced Oedema in Rat Paw

A model of acute inflammation induced by intraplantar injection of 1.5 mg of carrageenin in rat paw was used. Administration of the compounds (2 mg/Kg) was by the intravenous route 5 minutes before oedema was induced: 3 hours later the animals were sacrificed and their paws were weighed as an indication of inflammation. The control animals received saline instead of the test compounds. The weight of the paws of the control animals was taken to correspond to 100% on the inflammation index.

Results are shown in Table 3.

TABLE 3

| Compound | /o vs controls |
|---|---|
| 201006 (Proendotel ®) | 79 |
| 203152 | 90 |
| 201261 | 86 |
| 201273 | 67 |
| 201326 | 58 |
| 291330 | 77 |
| Indomethacin | 57 |
| Methylprednisolone | 56 |
| Saline solution | 100 |

Test 4—Inhibition of the Release of Nitrite-Nitrate in Rat Plasma

The release of nitrite-nitrite was measured with Griess reagent in blood samples taken 8 hours after the administration of LPS (0.1 mg/Kg) i.v.

The animals were treated with the compounds by o.s. (25 mg/Kg) 30 minutes before and 3 hours after LPS. The control animals received saline instead of the test compounds. The values for the release of nitrite-nitrate were made to correspond to 100%. Results are reported in Table 4.

TABLE 4

| Compound | % vs controls |
|---|---|
| 201006 (Proendotel) | 99 |
| 203152 | 108 |
| 201261 | 98 |
| 201273 | 76 |
| 201326 | 62 |
| 201330 | Not determined |
| Betamethasone | 20 |
| Saline solution | 100 |

Test 5—Inhibition of the Formation of Superoxide anions Induced by f-MLP in Human Whole Blood

Samples of whole blood diluted with PBS Cytochrome C were incubated for 20 minutes at 37° C. with the test compounds or saline before adding the chermotactic agent f-MLP (0.1 μm/l) in the presence of cytochalasin B. After 20 minutes of activation with this agent, the samples were centrifuged and readings were taken of the supernatants with a spectrophotometer to assess the reduction in Cytochrome C. Table 5 reports the concentrations that are effective in inhibiting the loss of Cytochrome C by 50%.

TABLE 5

| Compound | CI₅₀ μM/l |
|---|---|
| 201006 (Proendotel) | 52 |
| 203152 | >100 |
| 201261 | 100 |
| 201273 | >100 |
| 201326 | >100 |
| 201330 | 36 |
| SOD (unit/ml) | 0.5 |

Test 6—Inhibition of Collagen-Induced Platelet Aggregation in Human Whole Blood

The aggregation event was analysed by counting the single non-aggregated platelets with a cell counter 5 minutes after adding collagen (1 μg/ml). In these working conditions, collagen induces platelet aggregation of over 80%. The test compounds or saline as control were preincubated for 1 minute before the collagen was added.

Table 6 reports the concentrations that inhibit platelet aggregation by 50% compared to saline.

TABLE 6

| Compound | CI₅₀ μM/l |
|---|---|
| PG12 | 0.05 |
| 201006 (Proendotel) | 35.0 |
| 203152 | >100 |
| 201261 | >100 |
| 201273 | >100 |
| 201326 | 78 |
| 201330 | 93 |

Test 7—Toxicity After a Single Intravenous Administration

Male CD-1 mice were used to assess the maximum non-lethal does (MNLD—the highest does at which no cases of mortality are observed) and the maximum tolerated does (MTD—does at which no evident or marked signs of tocicity or altered behaviour are observed). Results are shown in Table 7.

TABLE 7

| Compound | DMT (mg/Kg) | DMNL (mg(Kg) |
|---|---|---|
| 201006 (Proendotel) | 6.25 | 12.5 |
| 203152 | 50 | 200 |
| 201261 | 50 | 200 |
| 201273 | 25 | 100 |
| 201326 | 6.25 | 50 |
| 201330 | 25 | 50 |

Test 8—Mutagenesis (Mini-Ames Test)

The Mini-Ames test is a version of the traditional Ames test which involves the incubation of bacterial cells on 35-mm dishes instead of 100-mm dishes, and it requires a smaller quantity of product while maintaining the reliability of the result.

The test is conducted on two strains of Salmonella typhimurium requiring histidine (TA 98 and TA 100) and two strains of Escherichia coli requiring tryptophan (WP2 pKM101 and WP2 uvrA pKM1091).

The bacterial cells were exposed to different concentrations of the test compounds in the presence and absence of a microsomal liver enzyme preparation, to reveal any possible metabolite activity.

Mutagenic activity was determined as the ability of the test compound to induce a significant increase in the number of mutant clones compared to those developed spontaneously in the cultures with the control vehicle.

None of the tested compounds (203152, 201273, 201326 and 201330) proved to be mutagenic.

The compounds of the invention can be prepared by known methods (Claisen's condensation and rearrangement, Pechmann's condensation, Williamson's synthesis, Fischer's esterification). The coumarins are synthesised by condensation between a resorcin and a β-ketoester. They can then be alkylated by substituting the aromatic hydrogens by reaction with formaldehyde and an amine. Alternatively, it is possible to alkylate the free hydroxyl with an allyl and transpose it thermally onto the aromatic ring, or the hydroxyl can be alkylated with an alkylating agent or with a spacer (an alkyl dihalide or an alkyl halide substituted with an epoxy ring, an ester, an amine) that may then be further alkylated.

The synthesised product may undergo hydrogenation to reduce the double bond between positions 3 and 4, or the oxygen in position 2 can be substituted by reaction with Lawesson's reagent.

Some examples of the preparation of the coumarin derivatives according to the present invention are reported in the following:

### EXAMPLE 1

Preparation of the Compound FID 203152

Fifty grams of 4-methyl umbelliferone (Fluka 69580 MW 194.18−0.26 mol) is salified with 14.4 g of KOH in ethanol (1:1 mol/mol); the solvent is evaporated and the yellow residue, redissolved in 1000 ml of 2-butanone, is alkylated with 37 g of allyl bromide (MW 120.98−0.31 mol).

The reaction lasts 8 hours, after which the solvent is evaporated, and the residue redissolved in ethyl acetate, washed with 1N NaOH, crystallised from ethanol and vacuum-dried.

The resulting product is vacuum-heated to 200° C. till the accumulated heat indicates Claisen's transposition reaction. Once cooled, the mass is redissolved in ethyl acetate and the transposed product is extracted with 1N NaOH (4-methyl-7-hydroxy 6 or 8 allyl hymecromone) which is then precipitated with 1N HCl.

Alkylation and transposition are repeated, thus obtaining 4-methyl-7hydroxy-6,8-diallyl-hymecromone (yield 15 g MW 257.25).

The resulting hymecromone (0.058 mol) is refluxed overnight in 250 ml of ethanol with 3.5 g of paraformaldehyde (MW 30.03−0.12 mol) and 10.5 g of morpholine (MW 87.12−0.12 mol). The next day, solvent is evaporated off and the residue is purified by silica gel chromatography with a 70-30-5 eluent (methylene chloride-ethyl acetate-methanol); the pure fractions are concentrated and vacuum-dried (yield 8 g MW 395 0.02 mol).

The resulting 3-morpholinomethyl 4-methyl-7-hydroxy-6,8-diallyl-coumarin is salified with KOH in ethanol (1:1 mol/mol). The solvent is evaporated and the residue is refluxed in 2 butanone with 2 g of 1,3-dibromopropane (MW 201.9−0.01 mol) for 24 hours. The solvent is evaporated, the residue is redissolved in ethyl acetate, washed with 1N NaOH and extracted with 1N HCl. The aqueous solution is neutralised with NaHCO₃, extracted with methylene chloride and purified by silica gel chromatography with methylene chloride-methanol-ammonia 30% at a gradient of between 98-2-0.2 and 90-10-0.2.

The clean fractions are concentrated, redissolved in ethyl acetate and treated with HCl in ethanol till the Congo red indicator changes colour. The solvent is evaporated and the product is freeze-dried from water (yield 5.2 g MW 823.86).

### EXAMPLE 2

Preparation of the Compound FID 203251

Five grams of 3-diethylaminoethyl-4-methyl-7-hydroxy-coumarin (prepared as described in GB 1,914,053, Example 6) (0.0182 mol MW 275.35) is treated with KOH in ethanol (1:1 mol/mol), the solvent is evaporated and the residue is redissolved in 2-butanone with 3.4 g of 1-bromo-3-chloropropane (0.0218 mol MW 157.44) by refluxing overnight. The KBr salt is filtered off and the solvent is evaporated. The residue is redissolved in ethyl acetate and washed with 1N NaOH. The solvent is evaporated and the monoalkyl derivative is precipitated with n-hexane, then reacted with the 3-diethylaminoethyl-4WO methyl-7-hydroxy-8-chloro-coumarin (prepared as described in Italian patent 1,088,554, example 1) (5 g, 0.0161 mol MW 309.8). Twenty-four hours later, the salt is filtered off and the solvent is evaporated. The residue is redissolved in ethyl acetate, washed with 1N NaOH, concentrated and purified by silica gel chromatography with a 90-5-0.4 eluent (methylene chloride-methanol-ammonia 30%).

The clean fractions are concentrated, redissolved in ethyl acetate and treated with HCl in ethanol till the Congo red indicator changes colour, concentrated and crystallised from isopropanol (yield 5.2 g MW 698.13).

### EXAMPLE 3

Preparation of the Compound FID 201261

Ten grams of 3-diethylaminoethyl-4-methyl-7-hydroxy-coumarin (0.0363 mol MW 275.35) is salified with KOH in ethanol (1:1 mol/mol), the solvent is evaporated and the residue is redissolved in 2-butanone with 6.0 g of epibromohydrine (0.044 mol MW 136.98) by refluxing for 12 hours. The KBr salt is filtered off and the solvent is evaporated. The residue is redissolved in ethyl acetate and washed with 1N NaOH. The organic solution is dried, concentrated, precipitated with n-hexane and vacuum-dried. The residue is redissolved in water, added with 10.8 g of L-cysteine (0.0891 mol MW 121.16), the pH is adjusted to 9 and the mixture is left to react under stirring at 37° C. Twelve hours later, it is purified by silica gel chromatography with methylene chloride-methanol-ammonia 30% at a gradient of between 80-15-2 and 60-30-7

The clean fractions are concentrated, redissolved in ethanol saturated with HCl under stirring for one hour, evaporated and freeze-dried from water (yield 6.1 g MW 553.55).

### EXAMPLE 4

Preparation of the Compound FID 201273

Five grams of 3-diethylaminoethyl-4-methyl-7-hydroxy-coumarin (0.0182 mol MW 275.35) is salified with KOH in ethanol (1:1 mol/mol). The solvent is evaporated and the residue is redissolved in 2-butanone with 9.1 g of epoxyhexane (0.091 mol MW 100.16) and refluxed for 48 hours. The KBr salt is filtered off and the solvent is evaporated. The residue is redissolved in ethyl acetate, washed with 1N NaOH, concentrated and purified by silica gel chromatography with an eluent of methylene chloride-methanol-ammonia, 30%, at a gradient of between 98-2-0.2 and 90-5-0.4.

The clean fractions are concentrated, redissolved in ethyl acetate and treated with HCl in ethanol until the Congo red indicator changes colour, concentrated and crystallised from acetone-hexane 2:1 (yield 3.1 g MW 411.97).

### EXAMPLE 5

Preparation of the Compound FID 201302

In a 250-ml reactor fitted with a stirrer and a thermostat set at −5° C., 5 g of pyrogallol (0.0396 mol MW 126.11) and 15.4 g of β-keto-diethylaminoethylester (0.0673 mol MW 229.31) are mixed together. Twenty-five ml of 90% sulphuric acid is slowly added, drop by drop. After completion of the addition, the mixture is left at −5° C. for 2 hours, then at room temperature for 24 hours, subsequently added with 100 ml of ethyl acetate and the pH is adjusted to 12 with ammonia, cooling the reaction mixture. The precipitate is filtered, washed with acetone, dissolved in 1N HCl, neutralised with sodium carbonate and extracted with methylene chloride. Upon cooling, the pure product precipitates and is redissolved in chloroform-ethanol 2:1 and treated with HCl in ethanol till the Congo red indicator changes colour. After evaporation of the solvent, it is crystallised from 95% ethanol (yield 3.0 g MW 327.81).

### EXAMPLE 6

Preparation of the Compound FID 201310

Ten grams of lactose (0.0278 mol MW 360.32) is suspended in 51 g of acetic anhydride (0.5 mol MW 102.09) and added with 100 ml of pyridine anhydride, drop by drop. The mixture is reacted overnight, after which it is evaporated and purified by silica gel chromatography eluting with toluene-acetone 4:1. The pure fractions are evaporated and the resulting peracetyl-lactose is vacuum-dried, suspended in 120 ml of anhydrous N,N-dimethylformamide, added with 5.7 g of ammonium carbonate (0.059 mol MW 96.09) and reacted under stirring for 24 hours, then evaporated and purified on silica gel eluting with toluene-acetone 9:1.

The pure fractions are evaporated and the resulting hydroxyperacetyl-lactose is vacuum-dried, dissolved in 250 ml of anhydrous methylene chloride with 9.3 ml of trichloroacetonitrile (0.0642 mol MW 144.4) and treated with 0.5 g of sodium hydride (0.0214 mol MW 24), stirring for 3 hours. The mixture is concentrated and purified on silica gel eluting to a toluene-acetone gradient of between 9:1 and 7:3. The pure fractions are evaporated, the resulting peracetyl-lactose-trichloroacetamidate is vacuum-dried and reacted with 3.0 g of 3-diethylaminoethyl-4-methyl-7-hydroxy-coumarin in 100 ml of anhydrous methylene chloride, in the presence of activated molecular sieves. 3.2 g of boron trifluoride-ethyletherate (0.0109 mol MW 295.68) are added and the mixture is reacted for 2 hours, then filtered, washed with 1N NaOH, dried, concentrated and purified by silica gel chromatography with an eluent of methylene chloride-methanol-ammonia, 30%, at a gradient of between 90-5-0.4 and 80-20-0.4.

The clean fractions are concentrated, hydrolysed in 100 ml of 1N NaOH/tert-butanol 1:1, and neutralised with HCl. The pH is adjusted to 8 with ammonia and the mixture is extracted with chloroform/n-butanol 1:1.

The solvent is evaporated off and the residue is freeze-dried from water (yield 3.2 g MW 599.64).

### EXAMPLE 7

Preparation of the Compound FID 201326

5.6 g of 3-diethylaminoethyl-4-methyl-7-hydroxy-8-chloro-coumarin (0.018 mol MW 309.8) is refluxed in toluene with 9.1 g of 1,2-epoxyhexane in the presence of 2 g of basic allumina super 1 for 24 hours. The solid matter is filtered off, washing with 1N NaOH. The mixture is concentrated and purified by silica gel chromatography with an eluent of methylene chloride-methanol-ammonia, 30%, at a gradient of between 98-2-0.2 and 90-5-0.4.

The clean fractions are concentrated, redissolved in ethyl acetate and treated with HCl in ethanol until the Congo red indicator changes colour, filtered and crystallised from acetone (yield 1.6 g MW 446.42).

### EXAMPLE 8

Preparation of the Compound FID 201330

a) 5 g of 3-diethylaminoethyl-4-methyl-7-hydroxy-8-chloro-coumarin (0.016 mol MW 309.8) is salified with KOH in ethanol (1:1 mol/mol). The solvent is evaporated and the residue redissolved in 2-butanone is reacted with 2 g of ethyl chloroacetate (0.016 mol MW 122.55) by refluxing for 12 hours. The KBr salt is filtered off and the solvent is evaporated. The residue is redissolved in ethyl acetate, washed with 1N NaOH, concentrated and purified by silica gel chromatography with an eluent of methylene chloride-methanol-ammonia, 30%, at a gradient of between 98-2-0.2 and 90-5-0.4.

The clean fractions are concentrated, hydrolysed in HCl 2N under stirring for 40 hours, then filtered and vacuum-dried.

b) The dry filtered product is reacted in anhydrous methylene chloride with 5.1 g of N,N-dicyclohexylcarbodiimide (0.0248 mol MW 206.33) and 4.8 g of cyclohexylamine (0.048 mol MW 99.18). Twenty-four hours later the unreacted matter is filtered off, the solution is concentrated and purified by silica gel chromatography with an eluent of methylene chloride-methanol-ammonia, 30%, at a gradient of between 98-2-0.2 and 80-20-0.4, redissolved in ethyl acetate and treated with HCl in ethanol until the Congo red indicator changes colour. The precipitate is filtered and washed with acetone (yield 2.8 g MW 521.9).

### EXAMPLE 9

Preparation of the Compound FID 201345

The 7-(carboxymethyl)oxyderivative prepared from 5 g of 3-diethylaminoethyl-4-methyl-7-hydroxy-8-chloro-coumarin (0.016 mol MW 309.8), as described in example 8, is reacted in anhydrous methylene chloride with 15.4 g of N,N-dicyclohexylcarbodiimide (0.0744 mol MW 206.33) and 6.0 g of L-phenylanine ethylester (0.031 mol MW 193.25). After 48 hours, the unreacted substance is filtered off, the mixture is concentrated and purified by silica gel chromatography with an eluent of methylene chloride-methanol-ammonia, 30%, at a gradient of between 98-2-0.2 and 80-20-0.4.

The pure fractions are concentrated, reacted in 20 ml of 40% methylamine in water (0.1696 MW 31.06) for 24 hours, then concentrated and purified by silica gel chromatography with an eluent of methylene chloride-methanol-ammonia, 30%, at a gradient of between 95-5-0.2 and 90-10.0.4, redissolved in ethanol and treated with HCl in ethanol until the Congo red indicator changes colour.

The precipitate is filtered and crystallised from ethanol 96% (yield 2.2 g MW 564.51).

### EXAMPLE 10

Preparation of the Compound FID 201359

Ten grams of 3-diethylaminoethyl-4-methyl-7-hydroxy-coumarin (0.0363 mol MW 275.35) is salified with KOH in ethanol (1:1 mol/mol). The solvent is evaporated and the residue is refluxed in 2-butanone with 6.0 g of epibromhydrine (0.044 mol MW 136.98) for 12 hours. The KBr salt is filtered off and the solvent is evaporated. The residue is redissolved in ethyl acetate, washed with 1N NaOH, dried, concentrated, precipitated with n-hexane and vacuum-dried. The residue is redissolved in water and added with 14.5 g of N-acetyl L-cysteine (0.0891 mol MW 163.19), the pH is adjusted to 9 and the mixture is reacted under stirring at 37° C. Twelve hours later, it is freeze-dried and purified by silica gel chromatography with methylene chloride-methanol-ammonia, 30%, 80-25-5.

The clean fractions are concentrated and freeze-dried from HCl (yield 10.0 g MW 531.07).

### EXAMPLE 11

Preparation of the Compound FID 201366

a) Five grams of 3-diethylaminoethyl-4-methyl-7-hydroxy-8-chloro-coumarin (0.016 mol MW 309.8) is salified with KOH in ethanol (1:1 mol/mol). After evaporating the solvent, the residue is refluxed in 2-butanone with 2.7 g of 1-bromopropane (0.022 mol MW 123.01) for 4 hours. The KBr salt is filtered off and the solvent is evaporated. The residue is dissolved in ethyl acetate, washed with 1N NaOH, concentrated and purified by silica gel chromatography with an eluent of methylene chloride-methanol-ammonia, 30%, at a gradient of between 98-2-0.2 and 80-20-0.4.

The clean fractions are concentrated and vacuum-dried.

b) The dry residue is reacted in toluene at 25° C. with 4.0 g of methyl iodide (0.028 mol MW 141.94) for 24 hours. The mixture is concentrated and purified by silica gel chromatography with an eluent of methylene chloride-methanol-ammonia, 2N, 85-15-5.

The clean fractions are concentrated, redissolved in ethyl acetate and treated with HCl in ethanol until the Congo red indicator changes colour, then filtered and crystallised from acetone-ethanol 20:1 (yield 3.6 g MW 402.37).

### EXAMPLE 12

Preparation of the Compound FID 201368

Ten grams of 3-diethylaminoethyl-4-methyl-7-hydroxy-coumarin (0.0363 mol MW 275.35) is salified with KOH in ethanol (1:1 mol/mol). The solvent is evaporated and the residue is refluxed in 2-butanone with 6.0 g of epibromhydrine (0.044 mol MW 136.98) for 12 hours. The KBr salt is filtered off and the solvent is evaporated. The residue is redissolved in ethyl acetate and washed with 1N NaOH, dried, concentrated, precipitated with n-hexane and vacuum-dried. The residue is redissolved in toluene, added with 7 g of L-proline ethyl ester (0.038 mol MW 183.13) and reacted by refluxing for 20 hours. The mixture is concentrated and purified by silica gel chromatography with an eluent of methylene chloride-methanol-ammonia, 30%, at a gradient of between 95-5-0.4 and 90-10-0.4.

The pure fractions are evaporated and hydrolysed in 1N NaOH/methanol 1:1. Two hours later the pH is adjusted to 8-9 and the reaction mixture is extracted with chloroform/n-butanol 1:1. The product is concentrated and freeze-dried.

The residue is redissolved in pyridine/anhydrous methylene chloride and added with 10 g of N,N-dicyclohexylcarbodiimide. Four hours later, the solvent is evaporated and the residue purified by silica gel chromatography with an eluent of methylene chloride-methanol-ammonia, 30%, at a gradient of between 90-5-0.4 and 70-30-0.4.

The clean fractions are concentrated, redissolved in ethyl acetate and treated with HCl in ethanol until the Congo red indicator has changed colour, then concentrated and freeze-dried from water (yield 4.1 g MW 501.45).

### EXAMPLE 13

Preparation of the Compound FID 201370

The 7-propoxy derivative obtained as in example 11 a) from five grams of 3-diethylaminoethyl-4-methyl-7-hydroxy-8-chloro-coumarin (0.016 mol MW 309.8) is refluxed in toluene at 120° C. with 4.9 g of Lawesson's reagent [2,4-bis(4-methoxyphenyl)-1,3,2,4-dithiadiphosphoethane-2,4-disulfide] (0.012 mol MW 404.45) for 4 hours. The mixture is concentrated and purified by silica gel chromatography with methylene chloride-methanol-ammonia, 30%, 90-5-0.4.

The clean fractions are concentrated, redissolved in ethyl acetate and treated with HCl in ethanol until the Congo red indicator has changed colour, then concentrated and precipitated from acetone (yield 0.7 g MW 404.40).

### EXAMPLE 14

Preparation of the Compound FID 201373

The 7-propoxy derivative obtained as in example 11 a) from five grams of 3-diethylaminoethyl-4-methyl-7-hydroxy-8-chloro-coumarin (0.016 mol, MW 309.8) is reacted in methylene chloride at 25° C. with 2.7 g of 3-chloroperoxybenzoic acid (0.016 mol MW 404.45) for 2 hours. The mixture is concentrated and purified by silica gel chromatography with methylene chloride-methanol-ammonia, 30%, at a gradient of between 90-5-0.4 and 80-20-0.5.

The clean fractions are concentrated, redissolved in ethyl acetate and treated with HCl in ethanol until the Congo red indicator has changed colour, then concentrated and crystallised from acetone/n-hexane 1:1 (yield 4.3 g MW 404.34).

### EXAMPLE 15

Preparation of FID 201264

30.3 g of 3-diethylaminoethyl-4-phenyl-7-hydroxy-coumarin (0,0802 mols, M. W. 337.86) (prepared as described in GB 1,013,053, example 1) are salified with KOH in ethanol (1:1 mols/mole); solvent is evaporated off, the residue is dissolved in 150 ml of DMSO and added with 26.5 g of 1-bromopropanol-2 (0.1605 mols, M.W. 165.07). After 10 days under stirring at 50° C., 300 ml of toluene and 150 ml of water are added. The organic phase is washed with 1M NaOH, then concentrated to dryness under vacuum. The residue is purified by silica gel chromatography with eluent CH2Cl2-CH3OH-ammonia 30%, at a gradient of between 98-2-0.2 and 90-5-0.4. The clean fractions are concentrated to dryness, redissolved in ethyl acetate and treated with HCl in ethanol until the Congo red indicator has changed colour, then concentrated and freeze-dried from water (yield 13.5 g, M.W. 431.96).

All the products were characterised and their structures confirmed by NMR and FT-IR analysis.

1. Coumarin derivatives of formula (I): 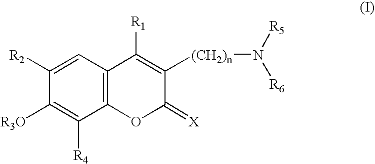
wherein:
X═O or S;
n=zero, 1, 2, 3 or 4;
R₅ and R₆, which can be the same or different, are optionally unsaturated C₁-C₄ alkyl groups, or together with the nitrogen atom they form a residue of cyclic amines optionally containing other heteroatoms;
R₁═CH₃ or phenyl;
R₂ and R₄, which can be the same or different, are H, OH, allyl, halogen or methyl;
R₃═H, straight or branched, saturated or unsaturated C₁-C₁₀ alkyl, which can bear OH groups, amido groups, residues of simple or derivatized sugars, or residues of optionally derivatized amino acids, or can be an optionally branched alkylene chain, (“spacer”), which links together the two residues, which can be the same or different, of formula (II) 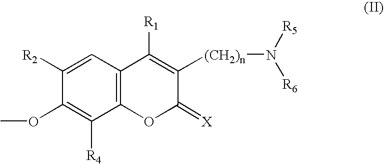
wherein X, n, R₁, R₂, R₄, R₅ and R₆ have the meanings defined above.

2. Coumarin derivatives as claimed in claim 1, selected from the group consisting of: 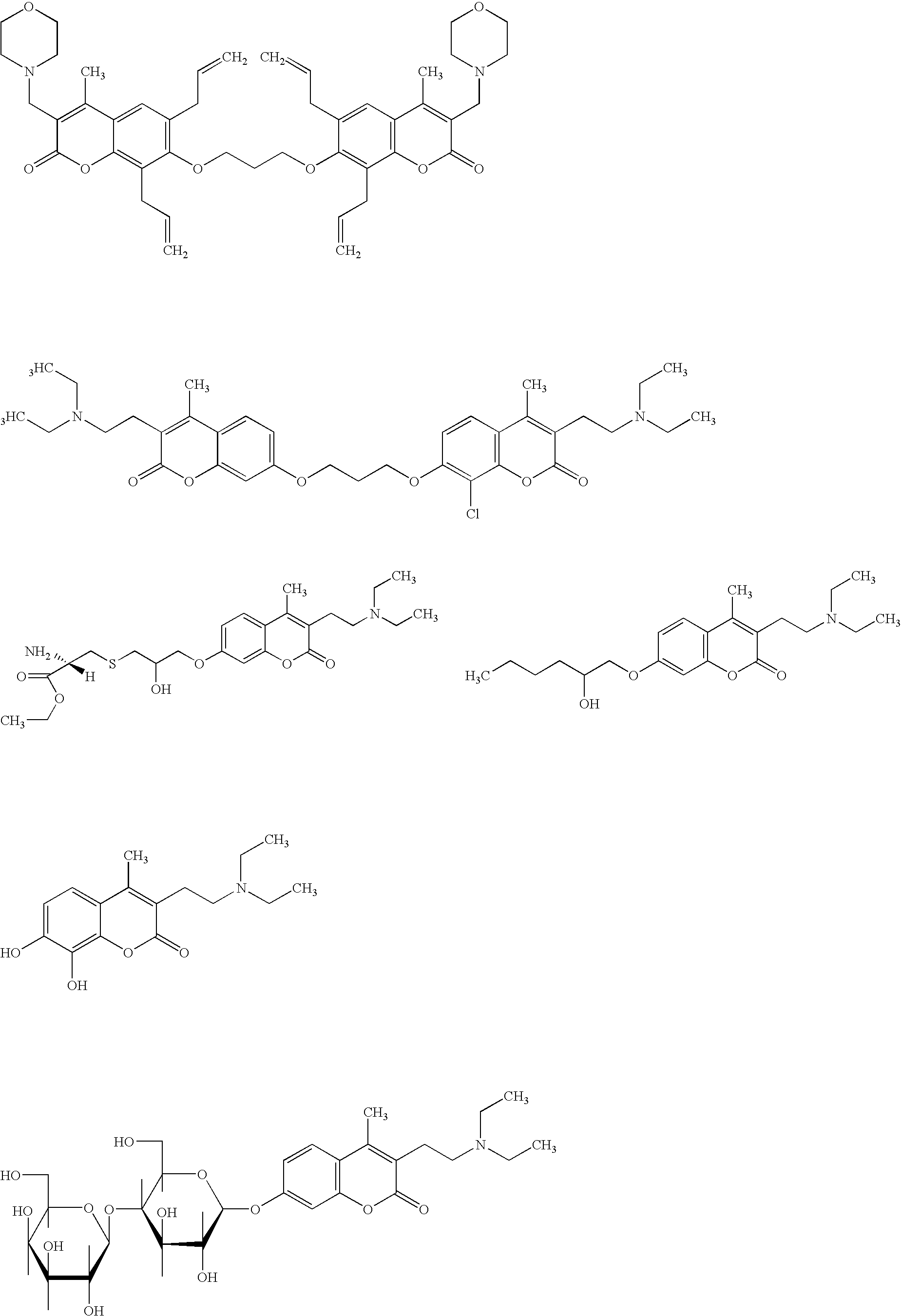 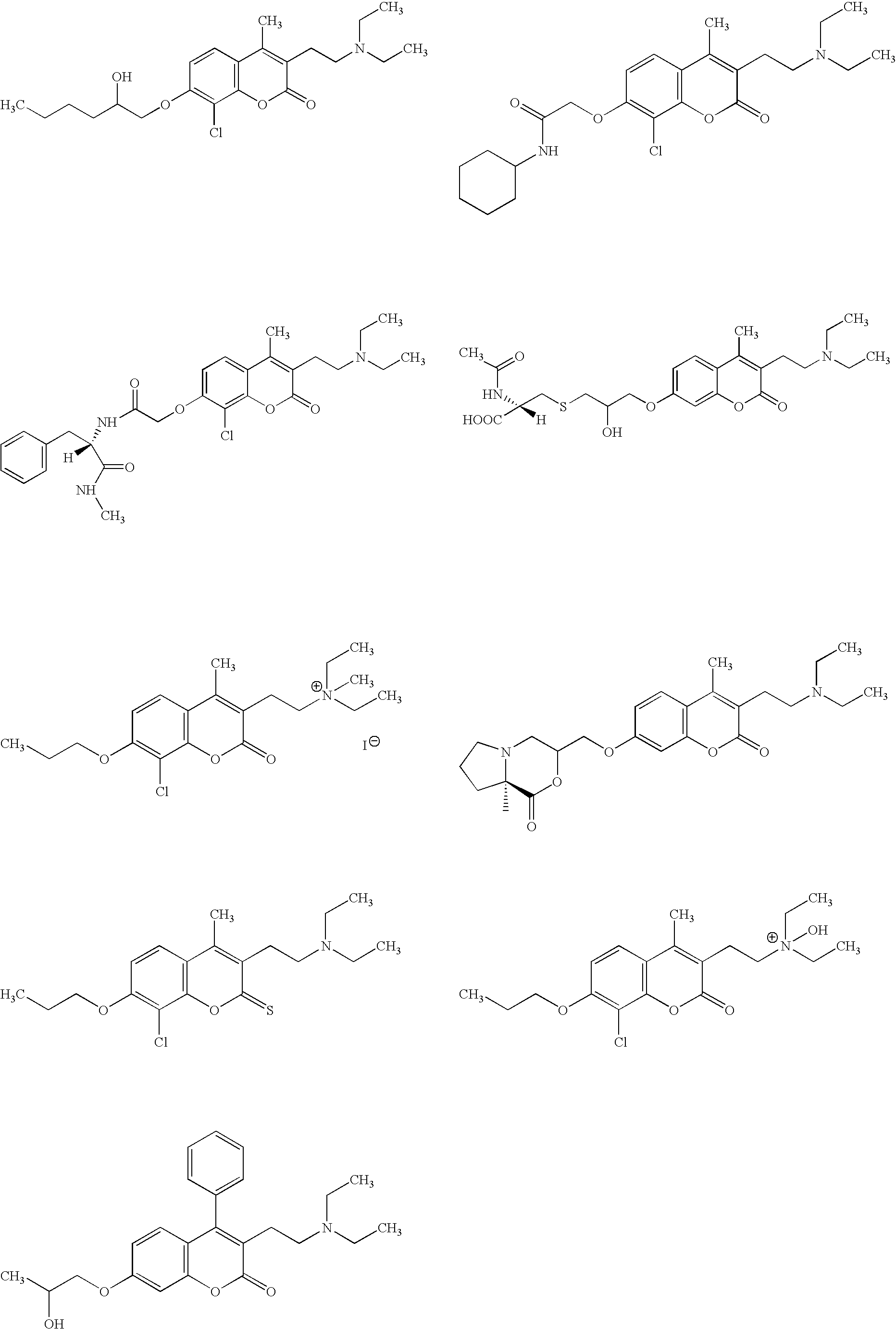
and the salts thereof with pharmaceutically acceptable acids or bases.

3. Pharmaceutical compositions as claimed in claim 3, in the form of capsules, tablets, injectable solutions, sprays, controlled release systems, creams, gels and transdermal systems.

4. Pharmaceutical compositions as claimed in claims 3 and 4, for the treatment of vascular (including those consequent on the release of proinflammatory molecules), dermatological and allergic pathologies, of hypercholesterolaemia and of systemic infections.

5. Pharmaceutical compositions as claimed in claims 3 and 4, for the treatment of peripheral vasculopathies, angina-type disorders and cerebral vasculopathies, peripheral ischaemia and ischaemia of organs.

6. Pharmaceutical compositions as claimed in claims 3 and 4, for the treatment of thrombosis and hypertension.
